(19) United States
(12) Patent Application Publication
(10) Pub. No.: US 2022/0071792 A1
(43) Pub. Date: Mar. 10, 2022

(54) JOINT FOR AN ORTHOPEDIC DEVICE

(71) Applicant: Ottobock SE & Co. KGAA, Duderstadt (DE)

(72) Inventors: David Brand, Duderstadt (DE); Martin Pusch, Duderstadt (DE)

(73) Assignee: Ottobock SE & Co. KGAA, Duderstadt (DE)

(21) Appl. No.: 17/416,360

(22) PCT Filed: Dec. 4, 2019
(86) PCT No.: PCT/EP2019/083624

§ 371 Date: Jun. 18, 2021

(30) Foreign Application Priority Data
Dec. 19, 2018 (DE) 10 2018 132 959.8

(51) Int. Cl. A61F 5/01

(52) U.S. Cl. CPC A61F 5/0123; A61F 2005/0148; A61F 2005/0167

(57) ABSTRACT

The invention relates to a joint for an orthopedic device, wherein the joint comprises a first element 2 and a second element 6, which are connected to one another via a connection device 10, wherein the connection device 10 is designed in such a way that the first element 2 can be swivelled relative to the second element 6 in a swivel range in a plane of movement, and can be displaced in a movement range in the plane of movement, and cannot be moved in directions outside of the plane of movement.

The invention relates to a joint for an orthopedic device, the joint having a first element and a second element that are connected to each other via a connection device. Advantageously, the first element and the second element are each provided to be connected to another component of the orthopedic device in which the joint is used.

It is necessary for a variety of different orthopedic devices, especially orthoses, to connect these different components of the orthopedic device in an articulated manner. Particularly in the case of orthoses, body parts, for example joints, are to be supported or exercised, so that for a variety of different applications it is desirable or advantageous if a swivelling of the two components of the orthopedic device relative to each other is possible, but a force must be overcome for this.

This can be achieved, for example, via damping elements, hydraulic arrangements, pneumatic arrangements or elastic elements.

One possible application of an orthopedic device, in particular an orthosis, is to restrict the movement range of a joint, for example a knee joint, in order to prevent for example, the ligaments present in the joint from being overstrained. Joints for orthopedic devices are also needed for this purpose.

In order to be able to ensure the best possible fit of the orthopedic device, which does not lead to pressure points or other loss of comfort regardless of the respective joint position of the joint of the wearer of the orthopedic device, it is advantageous if the swivel axis of the joint about which the two elements can be swivelled relative to each other coincides with the joint axis of the supported natural joint of the wearer of the orthopedic device. If this does not work, when the natural joint of the wearer of the orthopedic device is bent or stretched, the two components connected by the joint for the orthopedic device are displaced relative to the respective body parts.

Consequently, in a knee orthosis comprising an upper leg component for attachment to the upper leg and a lower leg component for attachment to the lower leg of the wearer connected by a joint for an orthopedic device, at least one of the two components would be displaced relative to the body part to which it is attached. On the one hand, this causes the bending of the joint to be uncomfortable for the wearer of the orthopedic device; on the other hand, it can lead to the orthopedic device no longer having the desired effect, or at least to the optimum extent.

The invention thus aims to further develop a joint for an orthopedic device in such a way that incongruities between the axis of the joint and that of the wearer's natural joint are compensated for in order to avoid or at least reduce the disadvantages described.

The invention solves the problem by way of a joint for an orthopedic device according to the preamble of claim 1, which is characterized in that the connection device is designed in such a way that the first element can be swivelled relative to the second element in a swivel range in a plane of movement, and can be displaced in a movement range in the plane of movement, and cannot be moved in directions outside of the plane of movement.

The configuration of the joint according to the invention ensures that the joint does not have a swivel axis that cannot be moved relative to the joint, as is known from the prior art, for example in the case of hinges or swivel joints. According to the invention, this is achieved by ensuring that the first element can not only be swivelled relative to the second element, but is also arranged such that it can be displaced in a movement range within the plane of movement. For example, if a joint according to the invention is incorporated into a knee orthosis and this knee orthosis is arranged on the wearer's leg in such a way that a swivel axis of the joint does not coincide with the natural swivel axis of the knee, this incongruity can be compensated for by displacing the first component relative to the second component.

It is especially preferable if this displacement occurs automatically without, for example, an orthopedic technician or the wearer of the orthopedic device themselves having to perform an adjustment or displacement. In particular, in the case of a knee joint, a joint described herein is advantageous for an orthopedic device, as the natural knee joint of the wearer also does not have a fixed and rigid swivel axis, but rather this swivel axis of the knee moves when the knee is bent and extended.

As with orthotic joints known from the prior art, swivelling the first element relative to the second element is only possible within a plane called the plane of movement in the joint for an orthopedic device described here. In conventional joints, this plane is perpendicular to the swivel axis. Swivelling is only possible within this plane of movement.

In this case, the joint is preferably configured in such a way that swivelling is only possible in a limited angular range, namely the swivel range. However, this lies entirely in the plane of movement.

The joint according to the invention is designed in such a way that the first element can be displaced relative to the second element within the plane of movement. Consequently, the first element can be moved relative to the second element in a straight motion towards or away from each other, wherein the direction of this motion is always within the plane of movement. Of course, non-straight movements are also possible, containing curves, for example, as long as the path of motion along which the first element is displaced relative to the second element lies entirely in the plane of movement.

While displacing the first element relative to the second element only changes the position of the two elements relative to each other, swivelling the first element relative to the second element essentially changes the orientation of the two elements relative to each other.

It is particularly preferable if the connection device of the joint is designed in such a way that simultaneous swivelling and displacement of the first element relative to the second element is also possible.

Regardless of the type of movement, the joint according to the invention is designed in such a way that movement outside the plane of movement is not possible. In the case of swivelling, this applies to swivel movements that are not perpendicular to the plane of movement, and in the case of displacement, to movements whose direction does not lie within the plane of movement. Such movements are not possible with joints according to the invention. Such movements prevented by the design of the joint do not include movements caused by backlash, production tolerances or corresponding inaccuracies.

In a preferred embodiment, the swivel range and/or the movement range is limited by the connection device. Here, the swivel range is an angular range in which the first element is swivelled relative to the second element. The movement range corresponds to the part of the plane of movement within which the two elements can be displaced relative to one another.

Advantageously, the first element features a first positive-locking element and the connection element a second positive-locking element which interact in way that restricts the range of movement. In a preferred embodiment, the first positive-locking element or the second positive-locking element is, for example, a slot, and the respective other positive-locking element a projection that engages in this slot, so that the projection slides along in the slot when the first element is displaced relative to the second element. Such slots are designed, for example, in the shape of an elongated hole or a bent or curved elongated hole, and feature a displacement contour that is determined by the shape and contour of the slot.

In a first embodiment, the first positive-locking element, which is arranged on the first element, is the slot. In this case, the connection device features the second positive-locking element in the form of the projection. In an alternative embodiment, the roles of the positive-locking elements are switched. In this case, the first positive-locking element on the first element is the projection, which engages in the second positive-locking element in the form of the slot on the connection device.

Of course, it is possible that the entire connection device or a part of the connection device is designed as a single piece with the second element.

By selecting the contour or shape of the slot and its length, the displacement range can be limited to a one-dimensional path, for example. This path does not have to be straight, but follows the contour of the slot.

In a preferred embodiment, the connection device comprises multiple positive-locking elements, each of which engages with the first positive-locking element. It is especially preferable if the at least two positive-locking elements can be moved relative to each other; preferably, they can be moved in the plane of movement.

Such an embodiment has, for example, two second positive-locking elements in the form of two projections. In this case, the first element comprises a first positive-locking element in the form of the slot. It is advantageous if the two second positive-locking elements can be moved relative to each other. For example, they can each be arranged on a component that can be displaced or moved relative to a base body of the connection device and/or relative to the second element. In this example, both second positive-locking elements engage in the slot which forms the first positive-locking element on the first element.

In an alternative configuration, the at least two second positive-locking elements are each designed in the form of a slot in which the one first positive-locking element on the first element in the form of a projection engages. In this case too, the at least two second positive-locking elements in the form of the slot are preferably arranged on separate components which can be displaced or moved relative to a base body of the connection device and/or relative to the second element.

In a structurally particularly simple configuration, only one of the two second positive-locking elements is arranged on such a separate component.

It is advantageous if the second positive-locking element is then also designed so that it can be moved relative to the second element if only one second positive-locking element is provided. This enables a two-dimensional movement range which can be determined and selected by the degree of movability of the second positive-locking element relative to a base body of the connection device and/or relative to the second element on the one hand, and the contour and length of the slot on the other.

In a preferred embodiment, the swivel range and/or the movement range is limited by a contour of the first element and the second element. For example, the two elements may each comprise a projection that abut against each other when the two elements reach a certain displacement position and/or a certain swivel position relative to each other, thereby preventing further movement in a certain direction or in multiple directions.

The connection device preferably has at least one elastic element, preferably at least one elastomeric element, which connects the first element and the second element. In this way, despite the considerable movability of the two elements relative to each other, a restoring force is applied as soon as the two elements are moved out of a rest position in which the elastic, preferably elastomeric, element is at its most relaxed.

In the following, some examples of embodiments of the present invention will be explained in more detail by way of the attached figures:

They show:

FIG. 1—the schematic representation of a joint according to an example of an embodiment of the present invention,

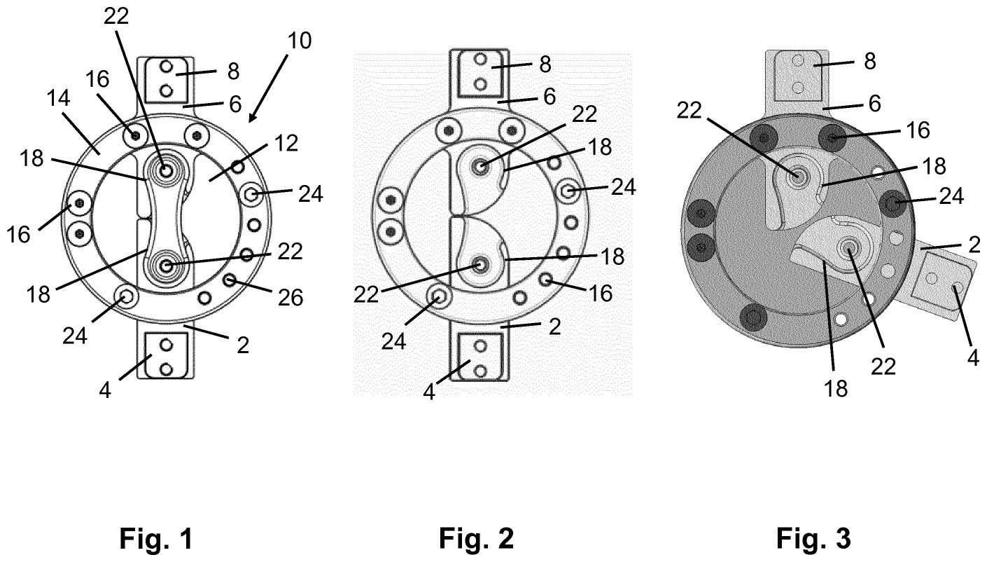

FIG. 2—the representation from FIG. 1 with no elastic element,

FIG. 3—the joint from FIG. 2 in the swivelled state,

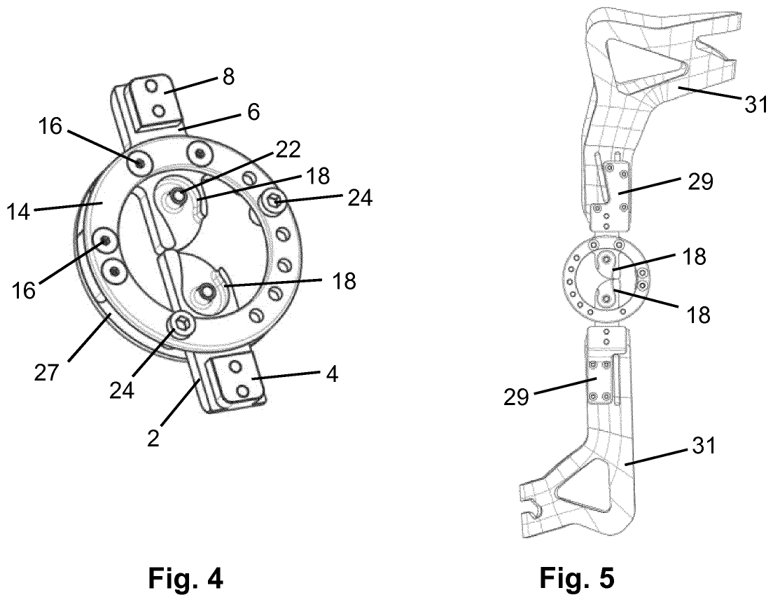

FIG. 4—the joint from FIGS. 2 and 3 in a schematic 3D view,

FIG. 5—the joint from FIG. 4 in an orthosis,

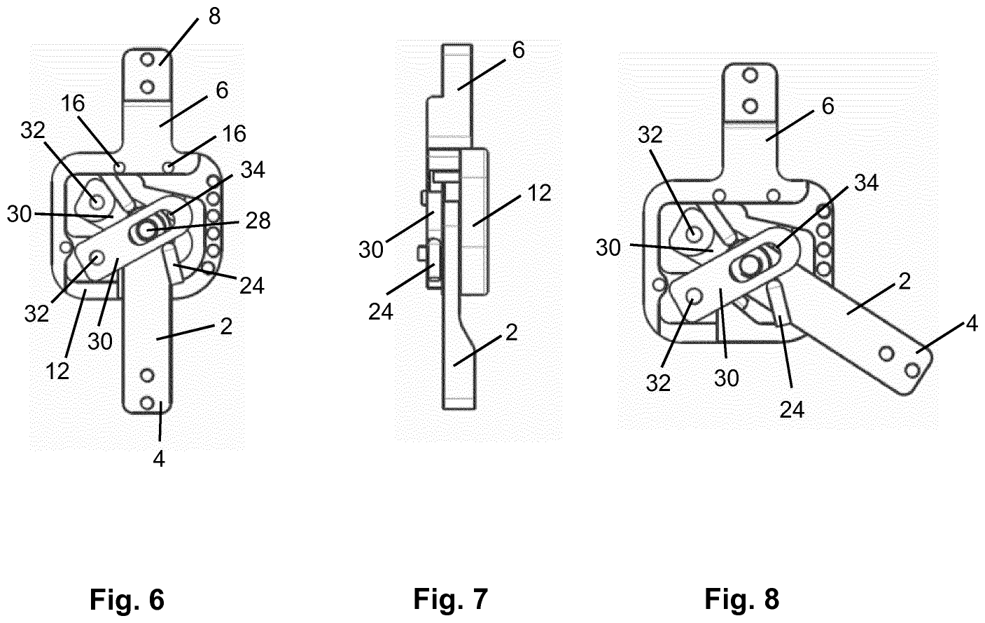

FIG. 6—the schematic depiction of a further example of an embodiment of the present invention,

FIG. 7—a side view of the joint from FIG. 6,

FIG. 8—the joint from FIGS. 6 and 7 in the swivelled state,

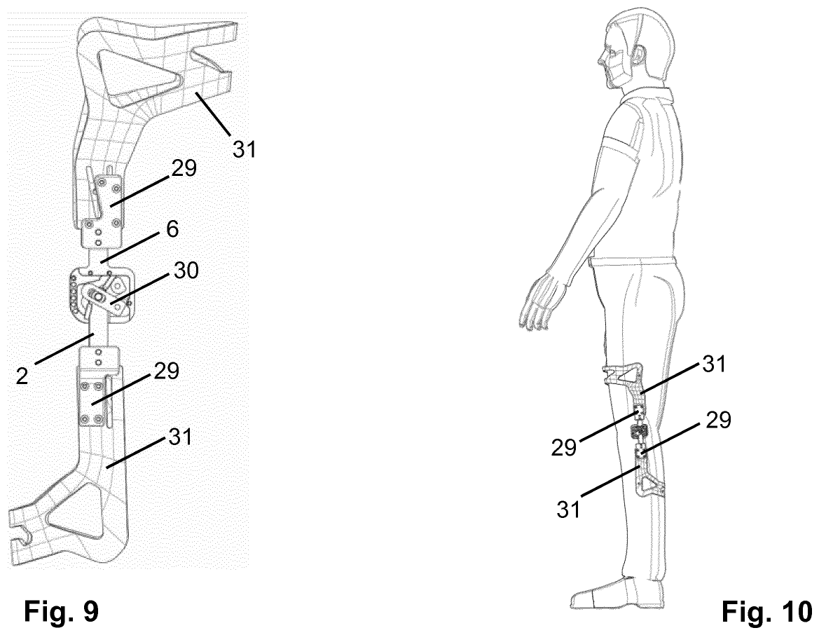

FIG. 9—the joint from FIGS. 6 to 8 in an orthosis,

FIG. 10—the orthosis from FIG. 9 in the mounted state,

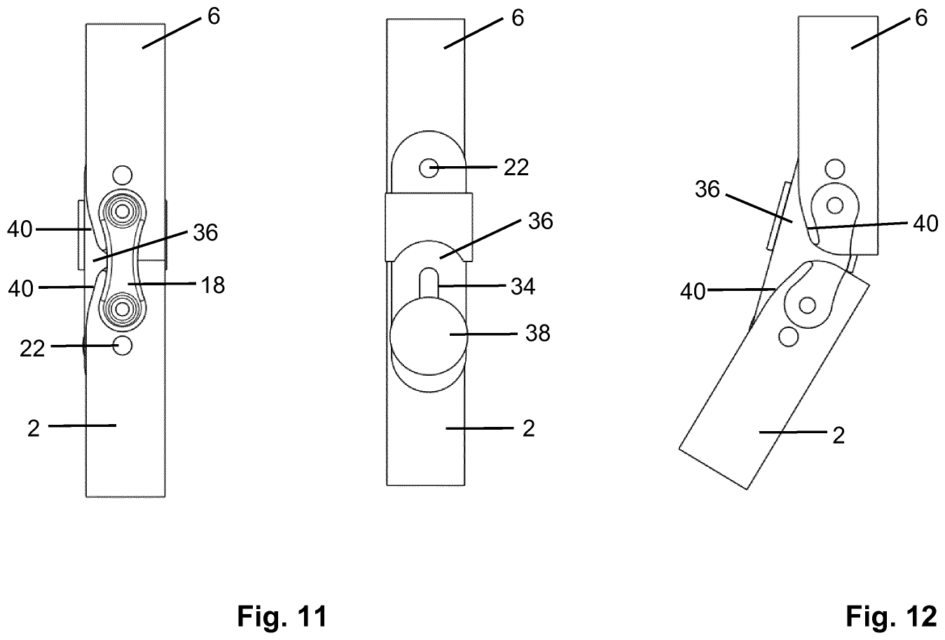

FIG. 11—the schematic depiction of a further example of an embodiment of the present invention,

FIG. 12—the joint from FIG. 11 in the swivelled state,

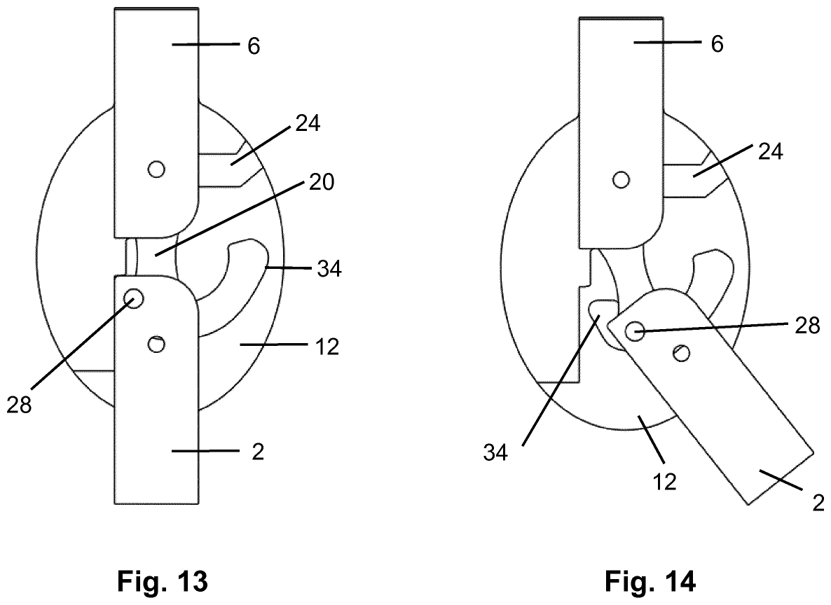

FIG. 13—the schematic depiction of a further example of an embodiment of the present invention,

FIG. 14—the joint from FIG. 13 in the swivelled state, and

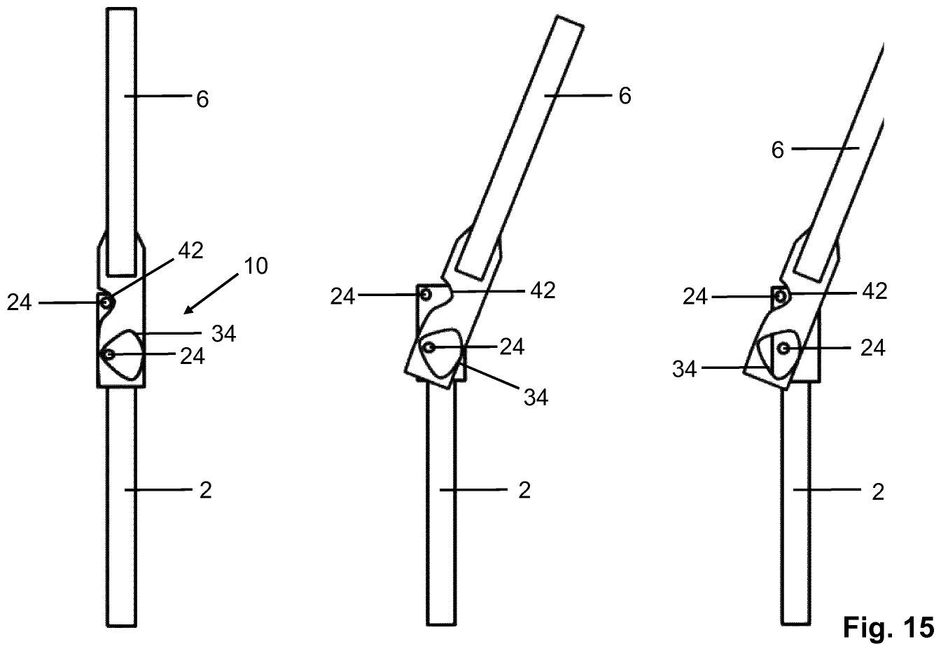

FIG. 15—a further embodiment of a joint.

FIG. 1 depicts a joint according to a first example of an embodiment of the present invention. The joint comprises a first element 2 with a first accommodation device 4 on which a first component, not depicted, of the orthopedic device can be arranged. The joint also comprises a second element 6 with a second accommodation device 8 on which a second component, also not depicted, of the orthopedic device can be arranged.

The components of the orthopedic device may be rails, for example, which lead to further components of the orthopedic device, such as fastening elements for attaching the device to the body of the wearer.

A connection device 10 is arranged on the second element 6, said connection device comprising a base body in the form of a rear plate and a guide ring 14. The second element 6 as well as the base body 12 and the guide ring 14 are connected to one another via screws 16. There is a circumferential gap between the base body 12 and the guide ring 14 through which the first element 2 projects.

Both the first element 2 and the second element 6 feature a receiving contour 18 into which an elastic element 20 is inserted. To this end, both the receiving contours 18 and two ends of the elastic element 20 each comprise a bore 22, through which screws can be guided in order to fix the elastic element 20 to the receiving contours 18 and thus to the first element 2 and the second element 6.

FIG. 2 shows the joint without the elastic element 20. The receiving contours 18 are particularly clear in this representation.

FIG. 3 depicts the representation from FIG. 2 in the swivelled state. In this position of the first element 2 relative to the second element 6, the elastic element 20, not depicted, has to be deformed. Due to the elastic properties of the elastic element 20, a restoring force occurs which counteracts further anti-clockwise swivelling of the first element 2 relative to the second element 6.

A swivel range about which the first element 2 can be swivelled relative to the second element 6 is limited by two end stops 24 which can be inserted into different passage openings 26, so that the swivel range can be adjusted.

Given that the elastic element 20 can be deformed, the first element 2 can be swivelled relative to the second element 6. However, since the elastic element 20 is also flexible, i.e. it can be stretched in the longitudinal direction, thereby increasing the distance between the two bores 22 of the elastic element 20, the first element 2 can also be displaced relative to the second element 6 in the longitudinal direction.

FIG. 4 shows the joint in a 3D view. The circumferential gap 27, through which the first element 2 protrudes, is particularly clear.

In FIG. 5, the joint is shown mounted in an orthosis. It is equipped with fastening elements 29, on which frame elements 31 are arranged, which can be arranged on the leg of a wearer in the example of an embodiment shown. Additional straps or belts that enclose the leg can be provided for attaching the frame elements 31 to the wearer's leg. These are not shown in FIG. 5.

FIG. 6 shows another configuration of a joint according to an example of an embodiment of the present invention. The first element 2 with the first accommodation device 4 and the second element 6 with the second accommodation device 8 are shown in the extended position in FIG. 6. The base body 12 is arranged on the second element 6 via the two screws 16. For the sake of better clarity, the guide ring 14 is not depicted.

Unlike in FIG. 1, the first element 2 has a first positive-locking element 28 in the form of a projection, peg or bolt, which protrudes upwards and downwards from the drawing plane in the example of an embodiment shown. Two components 30 are arranged on the base body 12, wherein said components can each be swivelled about a swivel axis 32. Each component 30 has a second positive-locking element 34 in the form of an elongated hole in which the first positive-locking 28 element engages. On the one hand, this allows a swivelling of the first element 2 relative to the second element 6. Since the components 30 are arranged such that they can be moved relative to the base body 12, the first element 2 can also be displaced relative to the second element 6. The size of the movement range is determined by the freedom of movement of the components 30 relative to the base body 12 as well as by the length of the two positive-locking elements 34 in the form of the elongated holes.

FIG. 7 depicts a side view of the joint from FIG. 6.

FIG. 8 shows the joint from FIGS. 6 and 7 in the swivelled state. It can be seen that the position of the components 30 relative to the base body 12 and thus also relative to the second element 6 has been slightly displaced. However, this is not absolutely necessary for swivelling the first element 2 relative to the second element 6.

As is the case in the embodiment shown in FIG. 6, there are two additional end stops 24 which restrict the freedom of movement of the upper component 30 with the second positive-locking element 34. This also limits the movement range in which the first element 2 can be displaced relative to the second element 6.

FIG. 9 shows the joint from FIGS. 6 to 8 in an orthosis. It is equipped with fastening elements 29, on which frame elements 31 are arranged, which can be arranged on the leg of a wearer in the example of an embodiment shown. Additional straps or belts that enclose the leg can be provided for attaching the frame elements 31 to the wearer's leg. These are not shown in FIG. 9.

FIG. 10 shows the orthosis in the fastened state. The frame elements are arranged on the wearer's upper and lower leg such that the joint is arranged in the knee area. Due to the configuration of the joint, it is not necessary to exactly align the swivel axis of the joint with the swivel axis of the human joint. Since the instantaneous axis of rotation of the knee changes and becomes displaced as the knee moves, this is not possible for the entire movement range anyway. Slight incongruities can be compensated for by way of the configurations of the joint described here.

FIG. 11 shows a further configuration of a joint according to an example of an embodiment of the present invention. The left-hand part of FIG. 11 shows a top view and the right-hand part of FIG. 11 depicts a view from below.

Both the first element 2 and the second element 6 feature a receiving contour 18 into which an elastic element 20, not depicted here, can be inserted. Screws, bolts or pegs can be guided through bores 22 in the first element 2 and the second element 6; a connection element 36 can be fixed to the first element 2 and the second element 6 via said screws, bolts or pegs. Like the elastic element 20, not depicted here, the connection element 36 forms part of the connection device 10. A peg that is guided through the lower bore 22 and on which a fastening plate 38 is located forms the first positive-locking element 28, which is guided through the second positive-locking element 34 in the form of the elongated hole. Consequently, in the position shown in FIG. 11, the first element 2 can be displaced up and down relative to the second element 6 by sliding the pin, on which the fastening plate 38 is arranged, in the second positive-locking element 34.

In the example of an embodiment shown, the first element 2 can also be swivelled relative to the second element 6, as a contour 40 of the first element 2 and the second element 6 allow as such. The maximum displacement of the first element 2 relative to the second element 6 is limited by the contours 40 on the one hand, and by the end stop 24 adjusted to fit the contour of the fastening plate on the other.

FIG. 12 depicts the joint from FIG. 11 in the swivelled state. Not only has the first element 2 been swivelled relative to the second element 6, but the first element 2 has also been displaced relative to the second element 6 due to the fact that a distance has emerged between the contours 40.

FIG. 13 shows a further embodiment of a joint according to an example of an embodiment of the present invention. The first element 2 is connected via the elastic element 20 to the second element 6, where the base body 12 of the connection device 10 is situated. The first positive-locking element 20 is shown on the first element 2, wherein the former protrudes downwards in the form of a peg or pin in the example of an embodiment shown and engages in the second positive-locking element 34 in the form of a slot. For the sake of better clarity, a guide ring 14 is not depicted. Due to the elastic element 20, the first element 2 can be both swivelled and displaced relative to the second element 6 when the first positive-locking element 28 slides within the second positive-locking element 34. An end stop 24 limits a possible swivel range and a possible movement range.

FIG. 14 depicts the joint from FIG. 13 in the swivelled state. The first positive-locking element 28 has been displaced to the right in the second positive-locking element 34, so that the first element 2 has been displaced relative to the second element 6. At the same time, the first element 2 is displaced relative to the second element 6. Since the first element 2 rests on the end stop 24, it can no longer be displaced or swivelled further in this direction.

FIG. 15 shows a further configuration of the joint in three different positions. In the far-left representation, the joint is extended. The first element 2 and the second element 6 are connected to each other via the connection device 10. The first element 2 is connected to two end stops 24 which strike corresponding components of the second element 6 when the first element 2 reaches predetermined positions and orientations relative to the second element 6. To this end, the outer contour of a part of the connection device 10 that is connected to the second element 6 features an indentation 42 in which one of the end stops 24 rests in the left-hand representation in FIG. 15. The second end stop 24 can also be considered a first positive-locking element that is arranged in a second positive-locking element 34 such that it can move freely. The first positive-locking element 28/the end stop 24 are designed in the form of a pin or peg and can move freely in the second positive-locking element, designed as a bore, recess or slot. The middle representation in FIG. 15 shows a swivelling of the second element 6 relative to the first element 2. The upper of the two end stops 24 no longer rests on the indentation 42 in the contour of the component on the second element 6. The position of the second end stop 24 relative to the second positive-locking element 34 remains almost unchanged. This is different in the right-hand image of FIG. 15. Here, the second element 6 has been displaced to the left relative to the first element 2 in FIG. 15.

However, it has only been slightly swivelled. The end stop 24 rests once again on the indentation 42, whereas the second end stop 24 is now no longer in contact with the edge of the second positive-locking element 34.

### REFERENCE LIST

2 first element
4 first accommodation device
6 second element
8 second accommodation device
10 connection device
12 base body
14 guide ring
16 screw
18 receiving contour
20 elastic element
22 bore
24 end stop
26 passage opening
27 circumferential gap
28 first positive-locking element
29 fastening element
30 component
31 frame element
32 swivel axis
34 second positive-locking element
36 connection element
38 fastening plate
40 contour
42 indentation

1. A joint for an orthopedic device, wherein the joint comprises
a first element (2) and
a second element (6),
which are connected to one another via a connection device (10), characterized in that the connection device (10) is designed in such a way that
the first element (2) can be swivelled relative to the second element (6) in a swivel range in a plane of movement, and
can be displaced in a movement range in the plane of movement, and
cannot be moved in directions outside of the plane of movement.

2. The joint according to claim 1, characterized in that the swivel range and/or the movement range is limited by the connection device (10).

3. The joint according to claim 1 or 2, characterized in that the first element (2) features a first positive-locking element (28) and the connection element (10) a second positive-locking element (34) which interact in way that limits the movement range.

4. The joint according to claim 3, characterized in that the first positive-locking element (28) or the second positive-locking element (34) is a slot, and the respective other positive-locking element (28, 34) a projection that engages in the slot, so that the projection slides along in the slot when the first element (2) is displaced relative to the second element (6).

5. The joint according to claim 3 or 4, characterized in that the connection device (19) comprises at least two positive-locking elements (34), each of which interacts with the first positive-locking element (10).

6. The joint according to claim 4 or 5, characterized in that the at least two second positive-locking elements (34) can be moved relative to each other; preferably, they can be moved in the plane of movement.

7. The joint according to claim 6, characterized in that the at least two second positive-locking elements (34) are arranged on two different components (30) of the connection device (10).

8. The joint according to one of the preceding claims, characterized in that the swivel range and/or the movement range is limited by a contour (40) of the first element (2) and the second element (6).

9. The joint according to one of the preceding claims, characterized in that the connection device (10) has at least one elastic element (20), preferably at least one elastomeric element, which connects the first element (2) and the second element (6).
